(19) United States
(12) Patent Application Publication
(10) Pub. No.: US 2021/0380867 A1
(43) Pub. Date: Dec. 9, 2021

(54) METHODS AND COMPOSITIONS FOR DOWNHOLE DIVERSION OF WELL TREATMENT FLUID

(71) Applicants: SAUDI ARABIAN OIL COMPANY, Dhahran (SA); KING FAHD UNIVERSITY OF PETROLEUM & MINERALS, Dhahran (SA)

(72) Inventors: Rajendra Arunkumar Kalgaonkar, Dhahran (SA); Khalid Alnoaimi, Aberdeen (GB); Nour Baqader, Khobar (SA); Nisar Ullah, Dhahran (SA); Muhammed Mansha, Dhahran (SA)

(73) Assignees: SAUDI ARABIAN OIL COMPANY, Dhahran (SA); KING FAHD UNIVERSITY OF PETROLEUM & MINERALS, Dhahran (SA)

(21) Appl. No.: 16/895,779

(22) Filed: Jun. 8, 2020

(51) Int. Cl. C09K 8/584; C07C 309/14; E21B 43/27

(52) U.S. Cl. CPC C09K 8/584; E21B 43/26; E21B 43/27; C07C 309/14

(57) ABSTRACT

In one aspect, embodiments disclosed herein relate to wellbore fluids that include a surfactant, calcium chloride, and an aqueous base fluid. The surfactant may have a structure represented by formula (I):

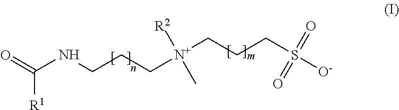

where R¹ is a C₁₅-C₂₇ hydrocarbon group, R² is a C₁-C₁₀ hydrocarbon group, and n and m are each, independently, an integer ranging from 1 to 4. The wellbore fluid may contain the calcium chloride in an amount of 5% by weight (wt. %) or more, relative to the total weight of the wellbore fluid.

Well stimulation enables the improved extraction of hydrocarbon reserves that conventional recovery processes, such as gas or water displacement, cannot access. One well stimulation technique is matrix stimulation, which may also be referred to as matrix acidizing treatment. In matrix stimulation, an acidic fluid is injected into a formation at a pressure below the fracture pressure and is used to stimulate a reservoir by reacting with the reservoir rock, thereby dissolving the rock to create a pathway for hydrocarbon production.

However, when the acidic fluid has a low viscosity, the acid may have limited penetration into the formation and only react at the face of the rock. This is not an effective method for stimulating the reservoir as a conductive pathway for hydrocarbon production is not created. Further, most of the reservoirs have heterogeneous permeabilities which result in the low viscosity acid primarily penetrating the high permeable zones in the reservoir and leaving most of the low permeability zones untreated.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to wellbore fluids that include a surfactant, calcium chloride, and an aqueous base fluid. The surfactant may have a structure represented by formula (I):

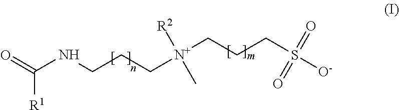

where R¹ is a C₁₅-C₂₇ hydrocarbon group, R² is a C₁-C₁₀ hydrocarbon group, and n and m are each, independently, an integer ranging from 1 to 4. The wellbore fluid may contain the calcium chloride in an amount of 5% by weight (wt. %) or more, relative to the total weight of the wellbore fluid.

In another aspect, embodiments disclosed herein relate to methods for treating a hydrocarbon-containing formation, the methods including injecting a wellbore fluid into a high permeability zone of a hydrocarbon-containing formation. The high permeability zone may increase the temperature of the wellbore fluid, resulting in the wellbore fluid having an increased viscosity. The wellbore fluids may include a surfactant, calcium chloride, and an aqueous base fluid. The surfactant may have a structure represented by the above formula (I). The wellbore fluid may contain the calcium chloride in an amount of 5% by weight (wt. %) or more, relative to the total weight of the wellbore fluid.

In another aspect, embodiments disclosed herein relate to methods for stimulating the recovery of hydrocarbons from a hydrocarbon-containing formation, the methods including injecting a wellbore fluid into a high permeability zone of a hydrocarbon-containing formation, stimulating the hydrocarbon-containing formation using the wellbore fluid thereby creating pathways for hydrocarbon production, and recovering the hydrocarbons. The high permeability zone may increase the temperature of the wellbore fluid, resulting in the wellbore fluid having an increased viscosity. The wellbore fluids may include a surfactant, calcium chloride, and an aqueous base fluid. The surfactant may have a structure represented by the above formula (I). The wellbore fluid may contain the calcium chloride in an amount of 5% by weight (wt. %) or more, relative to the total weight of the wellbore fluid.

In another aspect, embodiments disclosed herein relate to methods of preparing a wellbore fluid, the methods including mixing a surfactant, calcium chloride, and an aqueous base fluid. The surfactant may have a structure represented by the above formula (I). The wellbore fluid may contain the calcium chloride in an amount of 5% by weight (wt. %) or more, relative to the total weight of the wellbore fluid.

Other aspects and advantages of the claimed subject matter will be apparent from the following description and the appended claims.

### BRIEF DESCRIPTION OF DRAWINGS

FIG. 1 is schematic representation of the synthesis of a surfactant of one or more embodiments.

FIG. 2 is a flowchart depicting a well stimulation process in accordance with one or more embodiments of the present disclosure.

FIG. 3 is a schematic representation of the synthesis of an exemplary surfactant of one or more embodiments.

### DETAILED DESCRIPTION

Embodiments in accordance with the present disclosure generally relate to wellbore fluids that contain a surfactant and an activator, and methods of using the fluids in processes such as acid stimulation, enhanced oil recovery (EOR), and fracturing. The surfactant may be viscoelastic. Methods of one or more embodiments may involve injecting the wellbore fluids into a formation, exposing the fluid to an increased temperature and resulting in the wellbore fluid having an increased viscosity. Such methods may modify the injection profile of the formation a well stimulation treatment by diverting stimulation fluid to lower permeability zones of the reservoir.

The wellbore fluids may be low-viscosity aqueous solutions that increase in viscosity under downhole conditions. The wellbore fluids may demonstrate increased stability under high temperature and pressure conditions, making them highly suitable for use in downhole environments. When the wellbore fluid contacts a produced hydrocarbon its viscosity may drastically reduce, enabling easy flowback of the fluid post treatment. As the viscosifying material used in the present disclosure does not contain any solid particulates, it will be potentially non-damaging to the formation due to effective flowback and no residual deposition inside the formation.

The wellbore fluids of one or more embodiments of the present disclosure may include, for example, water-based wellbore fluids. The wellbore fluids may be acid stimulation fluids, EOR fluids, or fracturing fluids, among others.

In one or more embodiments, the water-based wellbore fluids may comprise an aqueous fluid. The aqueous fluid may include at least one of fresh water, seawater, brine, water-soluble organic compounds, and mixtures thereof. The aqueous fluid may contain fresh water formulated to contain various salts in addition to the first or second salt, to the extent that such salts do not impede the desired nitrogen-generating reaction. The salts may include, but are not limited to, alkali metal halides and hydroxides. In one or more embodiments, brine may be any of seawater, aqueous solutions wherein the salt concentration is less than that of seawater, or aqueous solutions wherein the salt concentration is greater than that of seawater. Salts that are found in seawater may include sodium, calcium, aluminum, magnesium, potassium, strontium, and lithium salts of halides, carbonates, chlorates, bromates, nitrates, oxides, phosphates, among others. Any of the aforementioned salts may be included in brine. In one or more embodiments, the density of the aqueous fluid may be controlled by increasing the salt concentration in the brine, though the maximum concentration is determined by the solubility of the salt. In particular embodiments, brine may include an alkali metal halide or carboxylate salt and/or alkaline earth metal carboxylate salts.

The wellbore fluids include a surfactant. In one or more embodiments, the surfactant may be a zwitterionic surfactant. The zwitterionic surfactant may be, for instance, derived from a betaine. In some embodiments, the zwitterionic surfactant may include a quaternary ammonium group and a sulfonate group. The zwitterionic surfactant of one or more embodiments may further comprise an amide group.

In one or more embodiments, the surfactant may have a structure represented by formula (I):

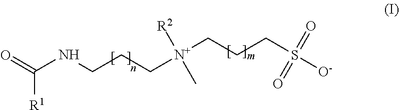

where R¹ is a C₁₅-C₂₇ hydrocarbon group or a C₁₅-C₂₉ substituted hydrocarbon group, R² is a C₁-C₁₀ hydrocarbon group, and n and m are each, independently, an integer ranging from 1 to 4.

As used herein with regard to groups R¹ and R², the term “hydrocarbon group” refers to branched, straight chain, and ring-containing hydrocarbon groups which may be saturated or unsaturated. The hydrocarbon groups may be primary, secondary, and/or tertiary hydrocarbons.

As used with regard to R¹, the term “substituted hydrocarbon group” refers to a hydrocarbon group (as defined above) where at least one hydrogen atom is replaced with a non-hydrogen group that results in a stable compound. Such substituents may be groups selected from, but not limited to, halo, hydroxyl, alkoxy, oxo, alkanoyl, aryloxy, alkanoyloxy, amino, alkylamino, arylamino, arylalkylamino, disubstituted amines, alkanylamino, aroylamino, aralkanoylamino, substituted alkanoylamino, substituted arylamino, substituted aralkanoylamino, thiol, alkylthio, arylthio, arylalkylthio, alkylthiono, arylthiono, substituted aryalkylthiono, alkylsulfonyl, arylsulfonyl, arylalkylsulfonyl, sulfonamide, substituted sulfonamide, nitro, cyano, carboxy, carbamyl, alkoxycarbonyl, aryl, substituted aryl, guanidine, and heterocyclyl, and mixtures thereof. In some embodiments, the substituted hydrocarbon group may comprise one or more alkylene oxide units. The alkylene oxide may be ethylene oxide.

In one or more embodiments, the zwitterionic surfactant may be soluble in aqueous solutions, such as in deionized water, seawater, brines, calcium chloride solutions, and the like. In some embodiments, the zwitterionic surfactant may be soluble in aqueous solutions in an amount of 10% by weight (wt. %) or more, 20 wt. % or more, or 30 wt. % or more at ambient temperature. In some embodiments, the solubility of the zwitterionic surfactant may increase with increasing temperature, until gelation occurs.

The wellbore fluids of one or more embodiments may comprise the surfactant in an amount of the range of about 1 to 15% by weight (wt. %). For example, the wellbore fluid may contain the surfactant in an amount ranging from a lower limit of any of 1, 1.5, 2, 2.5, 3, 4, 5, 7, 10, and 12 wt. % to an upper limit of any of 1.5, 2, 3, 4, 5, 6, 8, 10, 12, 14, and 15 wt. %, where any lower limit can be used in combination with any mathematically-compatible upper limit.

The wellbore fluids may include an activator. The activator is an additive that, upon an increase in temperature, enables the surfactant to exhibit viscoelastic behavior and cause the wellbore fluid to increase in viscosity. Without being bound by any theory, the activators disclosed herein may enable the surfactant micelles to form a rod-shaped structure that entangle as the temperature of the fluid increases. This entanglement is the cause of the viscoelastic behavior and the increase in viscosity.

In one or more embodiments, the activator may be a salt. The salt may, for instance comprise a monovalent cation, such as an alkali metal or a Group 11 transition metal, or a divalent cation, such as an alkaline earth metal or a transition metal. In some embodiments, the salt may comprise a cation selected from the group consisting of lithium, sodium, potassium, magnesium, calcium, nickel, iron, tin, aluminum, and zinc. In some embodiments, the salt may comprise an anion selected from the group consisting of fluoride, chloride, bromide, carbonate, bicarbonate, sulfate, nitrate, nitrite, chromate, sulfite, oxalate, phosphate, and phosphite. In particular embodiments, the activator may be an alkaline earth metal halide, such as calcium chloride.

The wellbore fluids of one or more embodiments may comprise the activator in an amount of the range of about 5 to 30% by weight (wt. %). For example, the wellbore fluid may contain the activator in an amount ranging from a lower limit of any of 5, 6, 7, 8, 10, 12, 15, 17, 20, and 22 wt. % to an upper limit of any of 10, 12, 15, 17, 20, 22, 25, 27, and 30 wt. %, where any lower limit can be used in combination with any mathematically-compatible upper limit.

In one or more embodiments, the wellbore fluid may comprise the activator and the surfactant in a weight ratio of 1:3 to 30:1, by weight, where the weight ratio is given as the weight of the activator to the weight of the surfactant. For example, the wellbore fluid may contain the activator and the surfactant in a weight ratio ranging from a lower limit of any of 1:3, 1:2, 1:1, 2:1, 4:1, 6:1, 8:1, 10:1, and 12:1 to an upper limit of any of 1:1, 2:1, 4:1, 6:1, 8:1, 10:1, 12:1, 20:1, and 30:1, where any lower limit can be used in combination with any mathematically-compatible upper limit.

The wellbore fluids of one or more embodiments may include one or more acids. Acids may be particularly included when the wellbore fluid is to be used in a matrix stimulation process, as described below. The acid may be any suitable acid known to a person of ordinary skill in the art, and its selection may be determined by the intended application of the fluid. In some embodiments, the acid may be one or more selected from the group consisting of hydrochloric acid, sulfuric acid, carboxylic acids such as acetic acid, and hydrofluoric acid. In some embodiments, the hydrofluoric acid may be included as a hydrogen fluoride source, such as ammonium fluoride, ammonium bifluoride, fluoroboric acid, hexafluorophosphoric acid, and the like.

The wellbore fluid of one or more embodiments may comprise the one or more acids in a total amount of the range of about 0.01 to 30.0 wt. %. For example, the wellbore fluid may contain the acids in an amount ranging from a lower limit of any of 0.01, 0.05, 0.1, 0.5, 1.0, 5.0, 10, 15, 20, and 25 wt. % to an upper limit of any of 0.5, 1.0, 5.0, 10, 15, 20, 25, and 30 wt. %, where any lower limit can be used in combination with any mathematically-compatible upper limit.

The wellbore fluids of one or more embodiments may include one or more additives. The additives may be any conventionally known and one of ordinary skill in the art will, with the benefit of this disclosure, appreciate that the selection of said additives will be dependent upon the intended application of the wellbore fluid. For instance, if the wellbore fluid is to be used as a fracturing fluid it may comprise a proppant, such as sand. In some embodiments, the additives may be one or more selected from clay stabilizers, scale inhibitors, corrosion inhibitors, biocides, friction reducers, thickeners, and the like.

The wellbore fluid of one or more embodiments may comprise the one or more additives in a total amount of the range of about 0.01 to 15.0 wt. %. For example, the wellbore fluid may contain the additives in an amount ranging from a lower limit of any of 0.01, 0.05, 0.1, 0.5, 1.0, 2.5, 5.0, 1.5, 10.0 and 12.5 wt. % to an upper limit of any of 0.1, 0.5, 1.0, 2.5, 5.0, 7.5, 10.0, 12.5, and 15.0 wt. %, where any lower limit can be used in combination with any mathematically-compatible upper limit.

In one or more embodiments, the wellbore fluid may contain little to no solid material. For example, the wellbore fluids of some embodiments may contain solid material in an amount of 2 wt. % or less, 1 wt. % or less, 0.5 wt. % or less, 0.1 wt. % or less, 0.05 wt. % or less, 0.01 wt. % or less, or 0.001 wt. % or less.

In one or more embodiments, the wellbore fluid may have a density that is greater than 0.90 g/cm³. For example, the wellbore fluid may have a density that is of an amount ranging from a lower limit of any of 0.90, 0.95, 1.00, 1.05, 1.10, 1.15, and 1.20 g/cm³ to an upper limit of any of 1.00, 1.05, 1.10, 1.15, 1.20, and 1.25 g/cm³, where any lower limit can be used in combination with any mathematically-compatible upper limit.

In one or more embodiments, the wellbore fluid may have a viscosity at 40° C. that is of the range of about 1 to 20 cP. For example, the wellbore fluid may have a viscosity at 40° C. that is of an amount ranging from a lower limit of any of 1, 2, 3, 4, 5, 6, 7, 8, 10, and 12 cP to an upper limit of any of 6, 8, 10, 12, 14, 16, 18, and 20 cP, where any lower limit can be used in combination with any mathematically-compatible upper limit. In some embodiments, the wellbore fluids may have a viscosity at 40° C. of 20 cP or less, 15 cP or less, or 10 cP or less.

In one or more embodiments, the wellbore fluid may have a viscosity at 90° C. that is of the range of about 20 to 150 cP. For example, the wellbore fluid may have a viscosity at 90° C. that is of an amount ranging from a lower limit of any of 20, 40, 60, 80, 100, and 120 cP to an upper limit of any of 30, 50, 70, 90, 110, 130, and 150 cP, where any lower limit can be used in combination with any mathematically-compatible upper limit. In some embodiments, the wellbore fluids may have a viscosity at 90° C. of 20 cP or more, 40 cP or more, 60 cP or more, 80 cP or more, 100 cP or more, or 120 cP or more.

In one or more embodiments, the wellbore fluid may have a ratio of a viscosity at 90° C. to a viscosity at 40° C. that is of the range of about 3:1 to 20:1. For example, the wellbore fluids may have a ratio of a viscosity at 90° C. to a viscosity at 40° C. that is of the range having a lower limit of any of 3:1, 4:1, 5:1, 6:1, 8:1, and 10:1 to an upper limit of any of 4:1, 5:1, 6:1, 8:1, 10:1, 12:1, 15:1, and 20:1, where any lower limit can be used in combination with any mathematically-compatible upper limit.

In one or more embodiments, the viscosity of the wellbore fluid may decrease after contacting with a hydrocarbon. For example, after contacting with a hydrocarbon such as diesel, the wellbore fluid may have a viscosity at 90° C. that is of an amount ranging from a lower limit of any of 1, 2, 3, 4, 5, 6, 7, 8, and 10, cP to an upper limit of any of 2, 4, 6, 8, 10, 12, and 15 cP, where any lower limit can be used in combination with any mathematically-compatible upper limit. In some embodiments, after contacting with a hydrocarbon such as diesel, the wellbore fluid may have a viscosity at 90° C. of 15 cP or less, 12 cP or less, 10 cP or less, 8 cP or less, or 5 cP or less.

In one or more embodiments, the wellbore fluid may have a pH that is neutral or acidic. For example, the wellbore fluid may have a pH ranging from a lower limit of any of 2, 3, 4, 4.5, 5, 5.5, and 6, to an upper limit of any of 3, 4, 4.5, 5, 5.5, 6, 6.5, and 7, where any lower limit can be used in combination with any mathematically-compatible upper limit. In some embodiments, the wellbore fluid may have a pH of 7 or less, of 6 or less, of 5 or less, of 4 or less, or of 3 or less.

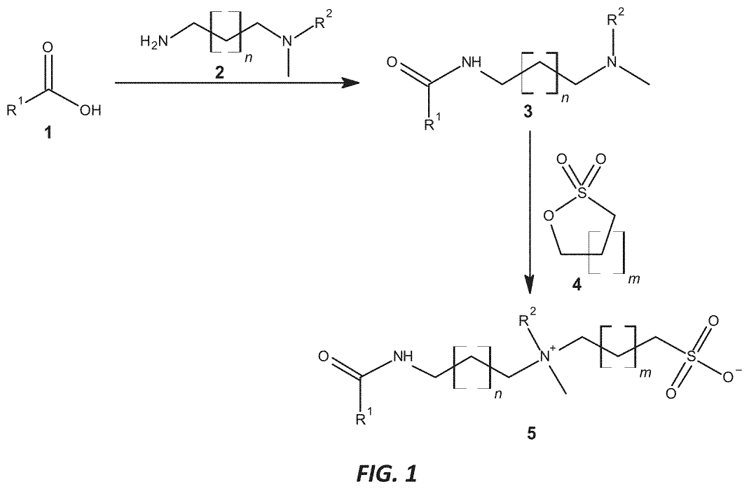

One or more embodiments of the present disclosure are directed to a synthesis of the surfactants represented by the aforementioned formula (I). A synthesis of one or more embodiments is depicted in FIG. 1, wherein R¹, R², n, and m represent the same groups as discussed above with regard to formula (I).

As shown by FIG. 1, a fatty acid 1 and an amine 2 may undergo an amidation reaction to yield intermediate amide 3. In one or more embodiments, an excess amount of amine 2 is used. In some embodiments, a molar ratio of amine 2 to fatty acid 1 is in a range of 1:1 to 5:1, or 2:1 to 4:1. In one or more embodiments, the amidation reaction may be performed at reflux. In some embodiments, the reaction is performed at a temperature that is of the range of 100 to 200° C., 140 to 180° C., or about 160° C. An external heat source, such as an oil bath, an oven, microwave, or a heating mantle, may be employed to heat the mixture to the aforementioned temperature. The mixture may be agitated throughout the duration of the reaction by any method known to a person of ordinary skill in the art, such as by employing a rotary shaker, a magnetic stirrer, or an overhead stirrer. In one or more embodiments, the amine 2 may be added in a two-stage or multi-stage fashion. For example, a first portion of the amine 2 of 50-70%, 55-65%, or about 57% of the total moles of the amine 2 used herein, may be added to the mixture and allowed to react with the carboxylic acid for 4-12 hours, 6-10 hours, or about 8 hours, and subsequently a second portion of the amine which is 30-50%, 35-45%, or about 43% of the total moles of the amine used herein may be added to the same mixture and allowed to react with the carboxylic acid for a duration of 3-9 hours, 5-7 hours, or about 6 hours. Alternatively, the amine may be introduced to the mixture in one batch and allowed to react with the carboxylic acid for 6-20 hours, 8-16 hours, or about 12 hours. The amidation reaction may be conducted under an inert atmosphere, such as under one or more of nitrogen, argon, and helium gas. After the reaction, the solid residue may be collected and washed with a solvent selected from one or more of the group consisting of acetone, water, ethyl acetate, and iso-propanol and subsequently dried under vacuum to yield amide 3 as a white solid. The intermediate amide 3 may be produced in a yield of 75% or more, 80% or more, 85% or more, 90% or more, 95% or more, or 97% or more.

In one or more embodiments, the reaction between fatty acid 1 and amine 2 may further include the use of a fluoride catalyst to facilitate the amidation. The fluoride catalyst may be one or more selected from the group consisting of sodium fluoride, potassium fluoride, silver fluoride, cesium fluoride, and tetrabutylammonium fluoride. A molar ratio of the fluoride catalyst to fatty acid 1 may be in a range of 1:5 to 1:20, 1:8 to 1:12, or about 1:10.

In one or more embodiments, the reaction vessel of the amidation reaction may further include a desiccant to facilitate the removal of any water produced during the reaction. The desiccant may be one or more selected from the group consisting of molecular sieves, alumina, anhydrous sodium sulfate, anhydrous magnesium sulfate, anhydrous calcium chloride, or anhydrous calcium sulfate. In some embodiments, the desiccant may be held in the reaction vessel separate from the reaction solution.

In alternative embodiments, a fatty acyl chloride may be used instead of fatty acid 1. In such instances, the reaction may be performed at a lower temperature, such as at 30° C. or less, 15° C. or less, or 5° C. or less.

Subsequently, as shown in FIG. 1, amide 3 may be reacted with a sultone 4 to yield a surfactant 5 having a structure consistent with formula (I). In one or more embodiments, a molar ratio of the sultone 4 to the amide 3 is in a range of 4:1 to 1:2, 3:1 to 1:1, or about 3:2. In some embodiments, the reaction may be performed with a molar excess of sultone 4. In one or more embodiments, this reaction is conducted in a polar aprotic solvent, such as one or more selected from the group consisting of ethyl acetate, dimethylformamide, tetrahydrofuran, acetone, acetonitrile, and dimethyl sulfoxide. In some embodiments, the reaction may be adapted to be performed in polar protic solvents such as one or more selected from the group consisting of methanol, ethanol, propanol, isopropyl alcohol, and butanol. In one or more embodiments, reacting the intermediate amide 3 with the sultone 4 is conducted without a solvent. In one or more embodiments, the reaction may be performed at reflux. In some embodiments, the reaction is performed at a temperature that is of the range of 50 to 100° C., 60 to 90° C., or about 80° C. In one or more embodiments, the reaction may have a duration of and has a reaction time of 2 to 36 hours, 2 to 24 hours, 4 to 16 hours, 10 to 14 hours, or about 12 hours. After the reaction, the solid residue may be collected and washed with a solvent selected from one or more of the group consisting of ethyl acetate and diethyl ether and subsequently dried under vacuum to yield surfactant 5 as a white solid. The surfactant 5 may be produced in a yield of 75% or more, 80% or more, 85% or more, 90% or more, 95% or more, or 97% or more.

Methods in accordance with the present disclosure may comprise the injection of a wellbore fluid into a formation. In one or more embodiments, the wellbore fluid may be a single treatment fluid that is injected into the wellbore in one pumping stage. In other embodiments, methods in accordance with one or more embodiments may involve the injection of the wellbore fluid and one or more additional stimulation fluids. The additional stimulation fluids may, in some embodiments, be co-injected with the wellbore fluid. In some embodiments, the stimulation fluids may be injected after the wellbore fluid.

The wellbore fluid of one or more embodiments has a low viscosity at low temperatures and, therefore, good injectivity, while being thermally stable enough for use downhole. Upon exposure to increased temperatures in the wellbore, the wellbore fluid may increase in viscosity. This phenomenon has the effect of reducing fluid mobility, resulting in diverting the flow from high permeability zones to lower ones and, ultimately, providing improved oil recovery.

The methods of one or more embodiments of the present disclosure may further comprise a pre-flushing step before the injection of the wellbore fluid. The pre-flushing step may comprise flushing the formation with a flushing solution that comprises a surfactant. The flushing solution may be an aqueous solution, and the surfactant may be the same surfactant as included in the wellbore fluid. The pre-flushing may limit the adsorption of the surfactant on the rock surface of the formation during the injection process. The suitability of the use of a pre-flushing step may depend on the type of surfactant and rock.

The hydrocarbon-containing formation of one or more embodiments may be a formation containing multiple zones of varying permeability. For instance, the formation may contain at least a zone having a relatively higher permeability and a zone having a relatively lower permeability. During conventional injection, fluids preferentially sweep the higher permeability zone, leaving the lower permeability zone incompletely swept. In one or more embodiments, the increased viscosity of the wellbore fluid may “plug” the higher permeability zone, allowing subsequent fluid to sweep the low permeability zone and improving sweep efficiency.

In one or more embodiments, the formation may have a temperature ranging from about 60 to 250° C. For example, the formation may have a temperature that is of an amount ranging from a lower limit of any of 60, 70, 80, 90, 100, 120, 140, 160, 180, and 200° C. to an upper limit of any of 100, 120, 140, 160, 180, 200, 225, and 250° C., where any lower limit can be used in combination with any mathematically-compatible upper limit.

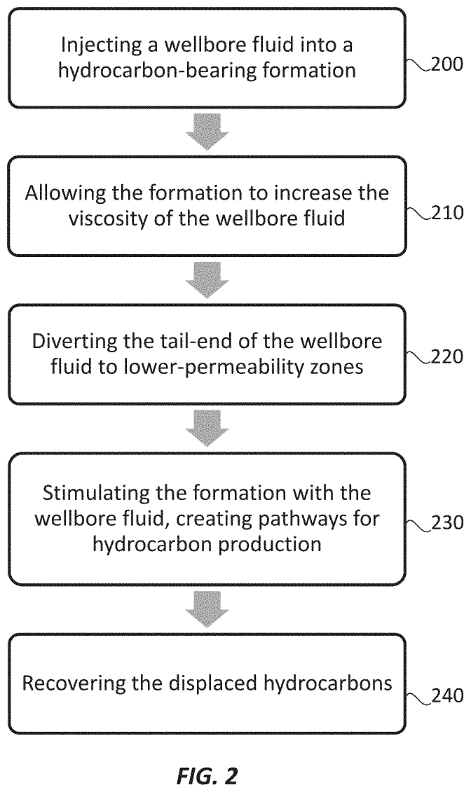

The methods of one or more embodiments may be used for well stimulation. A well stimulation process in accordance with one or more embodiments of the present disclosure is depicted by, and discussed with reference to, FIG. 2. Specifically, in step 200, the wellbore fluid may be injected into a hydrocarbon-bearing formation at an injection well. In some embodiments, the injection of the wellbore fluid may be performed at a pressure that is below the fracturing pressure of the formation. In step 210, a zone within the formation may be at a high temperature and increase the viscosity of the wellbore fluid. In step 220, after the increase in viscosity, the tail-end of the fluid is diverted to lower-permeability zones of the formation, displacing hydrocarbons. This results from the increase in viscosity that may “plug” the more permeable zones of the formation. In step 230, the formation is stimulated by the wellbore fluid, creating pathways for hydrocarbon production. In step 240, the displaced hydrocarbons may be recovered through the stimulated reservoir. In one or more embodiments, the hydrocarbons may be recovered at a production well.

The well stimulation process of one or more embodiments may be a matrix stimulation process. In the matrix stimulation process of one or more embodiments, the wellbore fluid, or one of the stimulation fluids, contains an acid. The acid fluid may react with the formation, dissolving rock, and creating wormholes that create a pathway for hydrocarbons to be displaced from deeper within the rock. In one or more embodiments, the wellbore fluid may increase in viscosity in the formation, enabling the fluid to better penetrate lower-permeability zones of the formation and allowing the acid to more uniformly react with the entire formation. This may provide for the formation of deeper wormholes and enhancing the overall permeability of the near-wellbore region. In the absence of this viscosity increase, the fluid will primarily penetrate the high permeability zones.

In one or more embodiments, the well stimulation process may be repeated one or more times to increase the amount of hydrocarbons recovered. In some embodiments, subsequent well stimulation processes may involve the use of different amounts of the surfactant and/or different surfactants than the first. The methods of one or more embodiments may advantageously provide improved sweep efficiency.

The methods of one or more embodiments may be used for fracturing a formation. In these embodiments, the wellbore fluid may be injected into a hydrocarbon-bearing formation at an injection well. A gas may be co-injected with the wellbore fluid to provide a foam. The foam may be driven through the formation at a pressure higher than the formation, opening pores and cracks present in the formation. The wellbore fluid of one or more embodiments may contain a proppant, such as sand, that can keep the pores and cracks of the formation open. These processes may, therefore, increase the permeability and hydrocarbon flow of the formation.

### EXAMPLES

The following examples are merely illustrative and should not be interpreted as limiting the scope of the present disclosure.

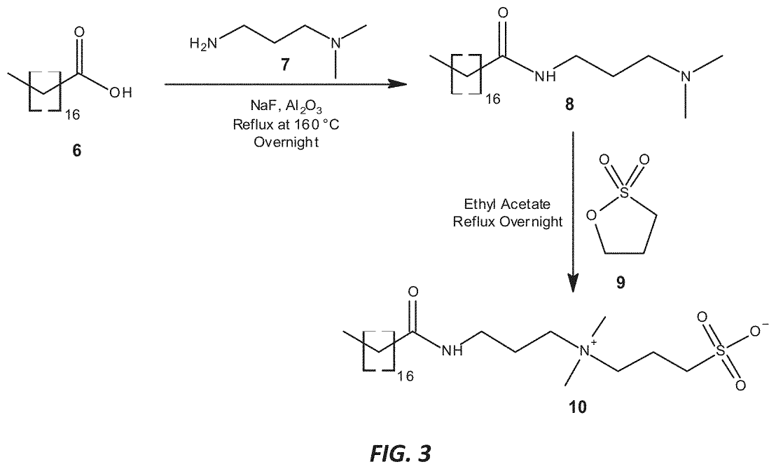

A zwitterionic surfactant (SDAS) 10 was prepared by the synthetic route illustrated in FIG. 3. Specifically, the SDAS 10 was synthesized by initially preparing the intermediate 8, and then then reacting 8 with 1,3-propane sultone 9.

### Synthesis of N-(3-(dimethylamino)propyl)nonadecanamide (8)

A two-necked round bottom flask, fixed with a reflux condenser and a bent tube, was charged with stearic acid 6 (5.00 g, 20.63 mmol), 3-(dimethylamino)-1-propylamine 7 (4.22 g, 41.25 mmol), and NaF (0.09 g, 2.06 mmol). The bent tube was filled with well dried alumina, which absorbs any water generated by the reaction. The flask was heated at a temperature of 160° C. for eight hours under a N2 atmosphere. A second aliquot of 3-(dimethylamino)-1-propylamine 7 (30.94 mmol) was added and the conditions were maintained for a further six hours. After cooling to room temperature, the solid residue was collected, washed with cold acetone: water (93:7 mL), and dried under vacuum to yield a white solid 8. ¹H-NMR [CD₃OD]=0.869 (t, 3H), 1.451-1.521 (m, 27H), 1.240-1.657 (m, 4H), 2.133 (t, 2H), 2.224 (s, 6H), 2.334 (t, 2H), 3.332 (t, 2H); ¹³C-NMR [CD₃OD]=18.95, 22.14, 25.22, 28.11, 33.32, 35.68, 35.88, 44.02, 50.66, 61.57, 63.46, 177.53, 180.53.

### Synthesis of 3-(metheyliumyl(methyl)(3-stearamidopropyl)-14-azaneyl)propane-1-sulfonate (SDAS, 10)

A 250-mL two-necked flask fixed with a reflux condenser was charged with 8 (5.00 g, 15.31 mmol), 1,3-propanesultone 9 (2.81 g, 22.97 mmol), and ethyl acetate (100 mL). The flask was heated at 80° C. for 12 h. After cooling to room temperature, the solid was collected, washed successively using ethyl acetate (100 mL) and diethyl ether (50 mL), and dried under vacuum to yield SDAS 10 as a white solid (6.14 g, 89% yield). ¹H-NMR [CDCl₃]=1.101 (t, 3H), 1.451-1.521 (m, 27H), 1.805 (m, 2H), 2.185 (t, 2H), 2.324-2.425 (m, 4H), 3.075 (t, 2H), 3.473 (t, 2H), 3.54 (s, 6H), 3.726 (t, 2H); ¹³C-NMR [CD₃OD]=14.0, 19.3, 22.6, 23.0, 25.9, 29.3, 29.6, 29.7, 31.9, 36.3, 36.4, 48.1, 50.9, 62.6, 63.2, 174.6; FTIR (cm⁻¹)=3265.42, 2915.00, 2884.61, 1666.49, 1552.64, 1467.54, 1174.26, 1035.13, 723.06.

Viscosification Experiments

The SDAS surfactant was mixed with two different concentrations of CaCl₂ in distilled water. The SDAS was used at concentrations of 2.5% (Examples 1 and 3) and 5% (Examples 2 and 4). The CaCl₂ were used at concentrations of 20% (Examples 1 and 2) and 30% (Examples 3 and 4) by weight. Thereafter, 2.5 wt. % or 5 wt. % of the SDAS was added to 95 wt. % or 97.5 wt. %, respectively, of the 20% or 30% CaCl₂ solutions. The viscosity of the surfactant solutions was measured at 40° C. and at 90° C. under different shear rates. The results are provided in Table 1

TABLE 1
Viscosity results for SDAS 10

| | Conc. of | CaCl₂ | CaCl₂ | Viscosity | Viscosity |
|---|---|---|---|---|---|
| | SDAS | (20%) | (30%) | (cPs; 40° C.) | (cPs; 90° C.) |
| Example 1 | 2.5% | 97.5% | — | 11.43 | 37.85 |
| Example 2 | 5% | 95% | — | 10.43 | 86.33 |
| Example 3 | 2.5% | — | 97.5% | 4.23 | 21.88 |
| Example 4 | 5% | — | 95% | 8.87 | 117.5 |

As is shown, under all conditions that were studied, the viscosity of the solution greatly increased upon heating to 90° C. Viscosity of the treatment fluid dropped to <10 cP after mixing with 10% by volume diesel at a temperature of 90° C.

The properties of Examples 1-4 indicate the suitability of surfactants such as SDAS 10 for use in wellbore fluids. These surfactants provide low-viscosity aqueous solutions that increase in viscosity under downhole conditions. When the wellbore fluid contacts a produced hydrocarbon its viscosity may drastically reduce, enabling easy flowback of the fluid post treatment.

Although the preceding description has been described herein with reference to particular means, materials and embodiments, it is not intended to be limited to the particulars disclosed herein; rather, it extends to all functionally equivalent structures, methods and uses, such as are within the scope of the appended claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112(f) for any limitations of any of the claims herein, except for those in which the claim expressly uses the words ‘means for’ together with an associated function.

What is claimed is:

1. A wellbore fluid, comprising:
a surfactant having a structure represented by formula (I): 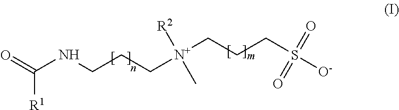
where R¹ is a C₁₅-C₂₇ hydrocarbon group, R² is a C₁-C₁₀ hydrocarbon group, and n and m are each, independently, an integer ranging from 1 to 4;
calcium chloride that is contained an amount of 5% by weight or more, relative to the total weight of the wellbore fluid; and
an aqueous base fluid.

2. The wellbore fluid according to claim 1, wherein the wellbore fluid contains the surfactant in an amount of 1 to 15 wt. %.

3. The wellbore fluid according to claim 1, wherein the wellbore fluid contains the activator in an amount of 10 to 30 wt. %.

4. The wellbore fluid according to claim 1, wherein the wellbore fluid further comprises an acid.

5. The wellbore fluid according to claim 1, wherein R¹ is a C₁₇ hydrocarbon group, R² is a C₁ hydrocarbon group, and n and m are both 1.

6. A method for treating a hydrocarbon-containing formation, comprising:
injecting a wellbore fluid into a high permeability zone of a hydrocarbon-containing formation, wherein the high permeability zone increases the temperature of the wellbore fluid, resulting in the wellbore fluid having an increased viscosity;
wherein the wellbore fluid comprises:
a surfactant having a structure represented by formula (I): 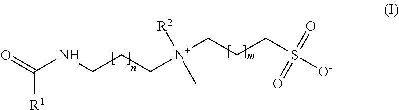
where R¹ is a C₁₅-C₂₇ hydrocarbon group, R² is a C₁-C₁₀ hydrocarbon group, and n and m are each, independently, an integer ranging from 1 to 4;
calcium chloride that is contained in an amount of 5% by weight or more, relative to the total weight of the wellbore fluid; and
an aqueous base fluid.

7. The method according to claim 6, wherein the wellbore fluid contains the surfactant in an amount of 1 to 15 wt. %.

8. The method according to claim 6, wherein the wellbore fluid contains the activator in an amount of 10 to 30 wt. %.

9. The method according to claim 6, wherein the wellbore fluid further comprises an acid.

10. The method according to claim 6, wherein R¹ is a C₁₇ hydrocarbon group, R² is a C₁ hydrocarbon group, and n and m are both 1.

11. A method for stimulating the recovery of hydrocarbons from a hydrocarbon-containing formation, the method comprising:
injecting a wellbore fluid into a high permeability zone of a hydrocarbon-containing formation, wherein the high permeability zone increases the temperature of the wellbore fluid, resulting in the wellbore fluid having an increased viscosity;
stimulating the hydrocarbon-containing formation using the wellbore fluid thereby creating pathways for hydrocarbon production; and
recovering the hydrocarbons,
wherein the wellbore fluid comprises:
a surfactant having a structure represented by formula (I): 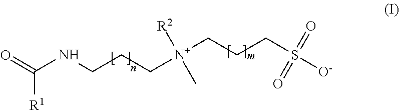
where R¹ is a C₁₅-C₂₇ hydrocarbon group, R² is a C₁-C₁₀ hydrocarbon group, and n and m are each, independently, an integer ranging from 1 to 4;
calcium chloride that is contained in an amount of 5% by weight or more, relative to the total weight of the wellbore fluid; and
an aqueous base fluid.

12. The method according to claim 11, wherein the wellbore fluid contains the surfactant in an amount of 1 to 15 wt. %.

13. The method according to claim 11, wherein the wellbore fluid contains the activator in an amount of 10 to 30 wt. %.

14. The method according to claim 11, wherein the wellbore fluid further comprises an acid.

15. The method according to claim 11, wherein R¹ is a C₁₇ hydrocarbon group, R² is a C₁ hydrocarbon group, and n and m are both 1.

16. The method according to claim 11, wherein the wellbore fluid contains the surfactant in an amount of 1 to 15 wt. %.

17. The method according to claim 11, wherein the displaced hydrocarbons contact the wellbore fluid, resulting in a decrease in a viscosity of the wellbore fluid.

18. A method of preparing a wellbore fluid, comprising:
mixing a surfactant, calcium chloride, and an aqueous base fluid,
wherein the surfactant has a structure represented by formula (I): 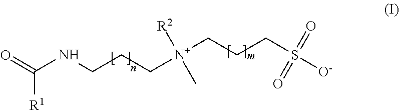
where R¹ is a C₁₅-C₂₇ hydrocarbon group, R² is a C₁-C₁₀ hydrocarbon group, and n and m are each, independently, an integer ranging from 1 to 4, and
wherein the wellbore fluid contains the calcium chloride in an amount of 5% by weight or more, relative to the total weight of the wellbore fluid.

19. The method according to claim 18, wherein the wellbore fluid contains the surfactant in an amount of 1 to 15 wt. %.

20. The method according to claim 18, wherein the wellbore fluid contains the activator in an amount of 10 to 30 wt. %.

21. The method according to claim 18, wherein the method further comprises mixing an acid with the surfactant, calcium chloride, and the aqueous base fluid.

22. The method according to claim 18, wherein R¹ is a C₁₇ hydrocarbon group, R² is a C₁ hydrocarbon group, and n and m are both 1.
